(19) United States
(12) Patent Application Publication
(10) Pub. No.: US 2009/0192813 A1
(43) Pub. Date: Jul. 30, 2009

(54) INFORMATION TRANSFER THROUGH OPTICAL CHARACTER RECOGNITION

(75) Inventors: Igor Gejdos, Indianapolis, IN (US); James R. Long, Fishers, IN (US); Timothy L. Beck, Pendleton, IN (US)

Correspondence Address: BAKER & DANIELS LLP / ROCHE, 300 NORTH MERIDIAN STREET, SUITE 2700, INDIANAPOLIS, IN, 46204, US

(73) Assignee: ROCHE DIAGNOSTICS OPERATIONS, INC., Indianapolis, IN (US)

(21) Appl. No.: 12/021,510

(22) Filed: Jan. 29, 2008

(51) Int. Cl. G06Q 99/00

(52) U.S. Cl. 705/1

(57) ABSTRACT

A medical data receiver configured to optically capture medical data, extract the medical data from the optical capture as alphanumeric characters, and provide the alphanumeric medical data for analysis.

### FIELD OF THE INVENTION

The present disclosure relates to a method and system for managing health data. More particularly, the disclosure relates a method and system for obtaining data.

### BACKGROUND OF THE INVENTION

Many fields of medical treatment and healthcare require monitoring of certain body functions, physical states and conditions, and patient behaviors. Thus, e.g., for patients suffering from diabetes, a regular check of the blood glucose level forms an essential part of the daily routine. The blood glucose level has to be determined quickly and reliably, often several times per day. Medical devices are used to facilitate the collection of medical information without unduly disturbing the lifestyle of the patient. A large number of medical devices for monitoring various body functions are commercially available. Also, medical treatment and healthcare may require monitoring of exercise, diet, meal times, stress, work schedules and other activities and behaviors.

To reduce the frequency of necessary visits to doctors, the idea of home care gained popularity over the recent years. Technological advancements in medicine led to the increased use of medical devices. Many of these medical devices, such as meters and medicine delivery devices, are able to collect and store measurements and other data for long periods of time. Other devices, such as computers, portable digital assistants (PDAs), and cell phones, have been adapted to medical uses by the development of software directed to the collection of healthcare data. These advancements led to the development of health management systems that enable collection and use of large numbers of variables and large amounts of healthcare data. While systems were traditionally developed for use in healthcare facilities and health management organizations including insurance companies and governmental agencies (HCP systems), increased technological sophistication by the populous at large led to the increased use of health management systems by patients, care givers, and others (patient systems) in addition to increased use by HCP systems. U.S. Pat. No. 7,103,578 and U.S. Published Application No. 2004/0172284 disclose two such methods and systems. Many of these systems are able to transfer data between them. Patient healthcare data is often transferred from a patient system to an HCP system. HCP systems may transfer remarks and other data to patient systems or other HCP systems.

### SUMMARY OF THE INVENTION

The disclosure relates to a medical data transmission system including a processor based image capturing device including instructions thereon. When the instructions are interpreted by the processor, they cause the processor to: capture an image of a display of a health management device having alphanumeric medical data thereon; perform optical character recognition on the captured image to extract the alphanumeric medical data from the image; and save the detected alphanumeric medical data.

In another embodiment, a computer readable medium is provided. The computer readable medium including operating instructions thereon such that when interpreted by a processor cause the processor to perform the steps of: capturing an image of a display of a health management device having alphanumeric medical information thereon; performing optical character recognition on the captured image to extract the alphanumeric medical information from the image as alphanumeric medical data; and saving the detected alphanumeric medical data.

In yet another embodiment, a medical data transmission system is disclosed including a wireless transceiver including a camera configured to capture an image of a display of a medical device having medical information thereon. The transceiver further includes a processor having instructions thereon to analyze the image and extract the medical information from the image as alphanumeric medical data in the form of alphanumeric characters. The transceiver further includes a transmitter to wirelessly emit the alphanumeric medical data.

In still another embodiment, a medical data transmission system is provided including: a first processor based image capturing device including instructions thereon that when interpreted by the processor cause the processor to: capture an image of a display of a health management device having alphanumeric medical data thereon; and transmit the captured image to a second processor based device programmed to perform optical character recognition on the captured image to extract the alphanumeric medical data from the image and save the detected alphanumeric medical data.

### BRIEF DESCRIPTION OF THE DRAWINGS

For more complete understanding of the present disclosure, reference is established to the following drawings in which:

FIG. 1 shows an embodiment of a health management system comprising a healthcare system and a homecare system;

FIG. 2 is a side view of a camera phone and a health management device that is part of the systems of FIG. 1;

FIG. 3 is a perspective view of the camera phone and health management device of FIG. 2;

Corresponding reference characters indicate corresponding parts throughout the several views. Although the drawings represent embodiments of various features and components according to the present invention, the drawings are not necessarily to scale and certain features may be exaggerated in order to better illustrate and explain the present invention. The exemplification set out herein illustrates embodiments of the invention, and such exemplifications are not to be construed as limiting the scope of the invention in any manner.

### DETAILED DESCRIPTION OF THE INVENTION

For the purposes of promoting an understanding of the principles of the disclosure, reference will now be made to the embodiments illustrated in the drawings, which are described below. The embodiments disclosed below are not intended to be exhaustive or limit the disclosure to the precise form disclosed in the following detailed description. Rather, the embodiments are chosen and described so that others skilled in the art may utilize their teachings. It will be understood that no limitation of the scope of the invention is thereby intended. The disclosure includes any alterations and further modifications in the illustrated devices and described methods and further applications of the principles of the disclosure which would normally occur to one skilled in the art to which the disclosure relates.

The terms “network,” “local area network,” “LAN,” “wide area network,” or “WAN” mean two or more computers which are connected in such a manner that messages may be transmitted between the computers. In such computer networks, typically one or more computers operate as a “server”, a computer with large storage devices such as hard disk drives and communication hardware to operate peripheral devices such as printers or modems. Other computers, termed “workstations”, provide a user interface so that users of computer networks can access the network resources, such as shared data files, common peripheral devices, and inter-workstation communication. The computers have at least one processor for executing machine instructions, and memory for storing instructions and other information. Many combinations of processing circuitry and information storing equipment are known by those of ordinary skill in these arts. A processor may be a microprocessor, a digital signal processor (“DSP”), a central processing unit (“CPU”), or other circuit or equivalent capable of interpreting instructions or performing logical actions on information. Memory includes both volatile and non-volatile memory, including temporary and cache, in electronic, magnetic, optical, printed, or other format used to store information. Users activate computer programs or network resources to create “processes” which include both the general operation of the computer program along with specific operating characteristics determined by input variables and its environment.

Concepts described below may be further explained in one of more of the previously-filed patent applications entitled HELP UTILITY FUNCTIONALITY AND ARCHITECTURE (Atty Docket: ROCHE-P0033), METHOD AND SYSTEM FOR GRAPHICALLY INDICATING MULTIPLE DATA VALUES (Atty Docket: ROCHE-P0039), SYSTEM AND METHOD FOR DATABASE INTEGRITY CHECKING (Atty Docket: ROCHE-P0056), METHOD AND SYSTEM FOR DATA SOURCE AND MODIFICATION TRACKING (Atty Docket: ROCHE-P0037), PATIENT-CENTRIC HEALTHCARE INFORMATION MAINTENANCE (Atty Docket: ROCHE-P0043), EXPORT FILE FORMAT WITH MANIFEST FOR ENHANCED DATA TRANSFER (Atty Docket: ROCHE-P0044), GRAPHIC ZOOM FUNCTIONALITY FOR A CUSTOM REPORT (Atty Docket: ROCHE-P0048), METHOD AND SYSTEM FOR SELECTIVE MERGING OF PATIENT DATA (Atty Docket: ROCHE-P0065), METHOD AND SYSTEM FOR PERSONAL MEDICAL DATA DATABASE MERGING (Atty Docket: ROCHE-P0066), METHOD AND SYSTEM FOR SETTING TIME BLOCKS (Atty Docket: ROCHE-P0054), METHOD AND SYSTEM FOR ENHANCED DATA TRANSFER (Atty Docket: ROCHE-P0042), COMMON EXTENSIBLE DATA EXCHANGE FORMAT (Atty Docket: ROCHE-P0036), METHOD OF CLONING SERVER INSTALLATION TO A NETWORK CLIENT (Atty Docket: ROCHE-P0035), METHOD AND SYSTEM FOR QUERYING A DATABASE (Atty Docket: ROCHE-P0049), METHOD AND SYSTEM FOR EVENT BASED DATA COMPARISON (Atty Docket: ROCHE-P0050), DYNAMIC COMMUNICATION STACK (Atty Docket: ROCHE-P0051), SYSTEM AND METHOD FOR REPORTING MEDICAL INFORMATION (Atty Docket: ROCHE-P0045), METHOD AND SYSTEM FOR MERGING EXTENSIBLE DATA INTO A DATABASE USING GLOBALLY UNIQUE IDENTIFIERS (Atty Docket: ROCHE-P0052), METHOD AND SYSTEM FOR ACTIVATING FEATURES AND FUNCTIONS OF A CONSOLIDATED SOFTWARE APPLICATION (Atty Docket: ROCHE-P0057), METHOD AND SYSTEM FOR CONFIGURING A CONSOLIDATED SOFTWARE APPLICATION (Atty Docket: ROCHE-P0058), METHOD AND SYSTEM FOR DATA SELECTION AND DISPLAY (Atty Docket: ROCHE-P0011), METHOD AND SYSTEM FOR ASSOCIATING DATABASE CONTENT FOR SECURITY ENHANCEMENT (Atty Docket: ROCHE-P0041), METHOD AND SYSTEM FOR CREATING REPORTS (Atty Docket: ROCHE-P0046), METHOD AND SYSTEM FOR CREATING USER-DEFINED OUTPUTS (Atty Docket: ROCHE-P0047), DATA DRIVEN COMMUNICATION PROTOCOL GRAMMAR (Atty Docket: ROCHE-P0055), HEALTHCARE MANAGEMENT SYSTEM HAVING IMPROVED PRINTING OF DISPLAY SCREEN INFORMATION (Atty Docket: ROCHE-P0031), and METHOD AND SYSTEM FOR MULTI-DEVICE COMMUNICATION (Atty Docket: ROCHE-P0064), the entire disclosures of which are hereby expressly incorporated herein by reference. It should be understood that the concepts described below may relate to diabetes management software systems for tracking and analyzing health data, such as, for example, the Accu-Chek® 360° product provided by Roche Diagnostics. However, the concepts described herein may also have applicability to apparatuses, methods, systems, and software in fields that are unrelated to healthcare. Furthermore, it should be understood that references in this patent application to devices, meters, monitors, pumps, or related terms are intended to encompass any currently existing or later developed apparatus that includes some or all of the features attributed to the referred to apparatus, including but not limited to the Accu-Chek® Active, Accu-Chek® Aviva, Accu-Chek® Compact, Accu-Chek® Compact Plus, Accu-Chek® Integra, Accu-Chek® Go, Accu-Chek® Performa, Accu-Chek® Spirit, Accu-Chek® D-Tron Plus, and Accu-Chek® Voicemate Plus, all provided by Roche Diagnostics or divisions thereof.

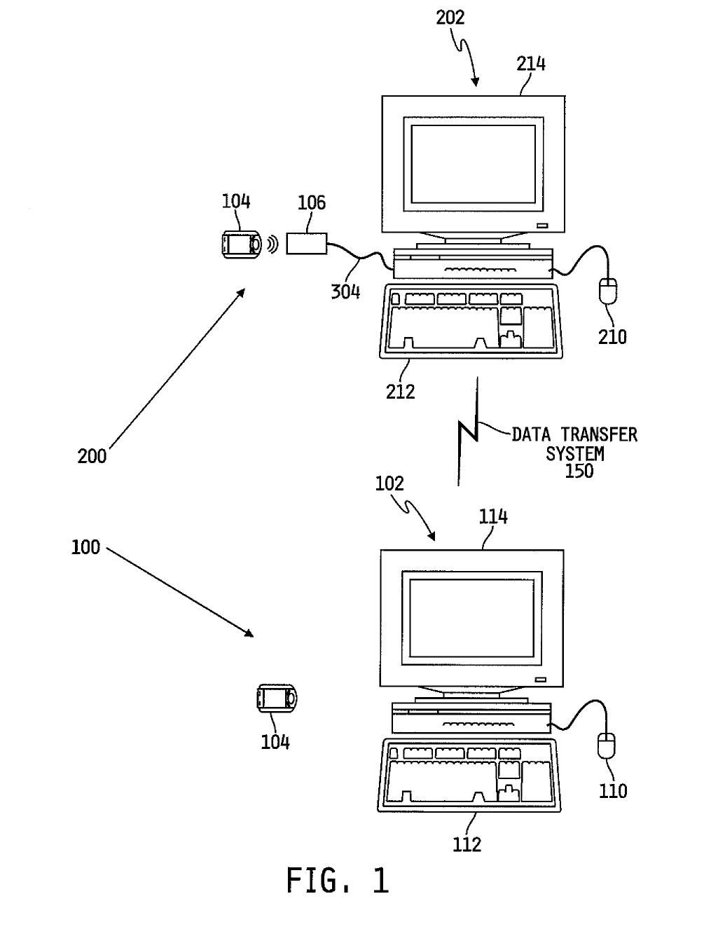

Turning now to the figures, FIG. 1 depicts an exemplary embodiment of a homecare system 100 and healthcare system 200 connected via a WAN 150 for monitoring data. Systems 100, 200 each comprise a computing device, shown here in the form of computers 102, 202 having processing units, system memory, display devices 114, 214, and input devices 112, 212, 110, 210, 106. Healthcare computer 202 may be, but is not necessarily, acting as a server. Furthermore, while only two computers 102, 202 are shown, many more computers may be part of the overall system.

While standard input devices such as mice 110, 210 and keyboards 112, 212 are shown, systems 100, 200 may comprise any user input device. By example, infrared (IR) dongle 106 is coupled to computer 202. IR dongle 106 is configured to send and receive IR transmissions from health management device 104. Computer 202 includes software applications configured to receive data from health management device 104 via IR dongle 106 or otherwise.

While computer 202 includes dongle 106 that is able to receive IR signals, some computers, such as computer 102, do not include IR communication means. While communications and health management devices 104 are discussed that make use of IR, the present disclosure is intended to cover devices and computers that communicate through other means. Accordingly, like computer 102, sometimes communication paths that are native to a selected health management device 104 are not available on the computer 102 that a user wishes to utilize. Additionally, a user may have a computer 102 that is able to communicate with his/her health management device, but the user may be at a location away from the computer 102 at the time that the user wishes to report measurement data.

Systems 100, 200 include health management software (not shown) configured to receive medical information from one or more of input devices 112, 212, 110, 210, 106. Health management devices 104 are described herein as meters, but could also be a PDA, therapeutic pump, combinations thereof, or other devices that store medical data thereon. Medical information may include blood glucose values, A1c values, Albumin values, Albumin excretion values, body mass index values, blood pressure values, carbohydrate values, cholesterol values (total, HDL, LDL, ratio) creatinine values, fructosamine values, HbAl values, height values, insulin dose values, insulin rate values, total daily insulin values, ketone values, microalbumin values, proteinuria values, heart rate values, temperature values, triglyceride values, weight values, and any other medical information that is desired to be known.

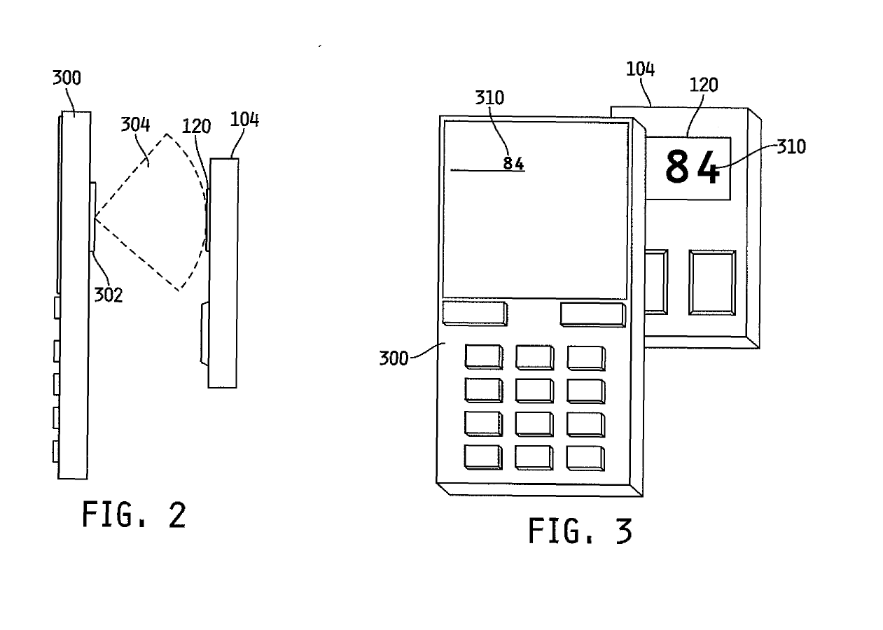

As shown in FIG. 2, health management device 104 includes screen 120 that displays the various medical information thereon as well as information about when the reading was taken. If a user is near computer 102, the user can manually input the displayed data into the health management software on computer 102 via keyboard 112, mouse 110, and the like. Such manual input of information results in the saved data being tagged as manually input. This tagging provides that a health care professional reviewing the data at a later time knows that the entry was manual. Manual entries into the health management software are treated as less reliable than electronically conveyed entries in that manual entries provide an increased potential to be accidentally incorrect (such as through a transcription error) or purposefully incorrect (such as to hide poor patient compliance). In addition to being more reliable, electronically conveyed entries can be easier for the user and can be automated.

Mobile device 300, FIG. 2, is illustratively a cell phone that includes camera 302. However, embodiments are envisioned for other devices that are able to take pictures such as computers with webcams (either integrated or add on) or cameras that can transfer files to computer 102 via a wired connection, removable memory, or otherwise. Mobile device 300 includes optical character recognition software that interfaces with camera 302. Alternative embodiments are discussed below where optical character recognition software instead resides on computer 102 or another computing device such as, but not limited to, a PDA with therapy advising software thereon.

In use, health management device 104 is first used to obtain a measurement. Screen 120 displays the measurement information. Mobile device 300 is aligned, by a user, with health management device 104 such that screen 120 is within a capture range 304 of camera 302. The user then activates an image capture with camera 302. Alternatively, software may be present on mobile device 300 to automatically perform the image capture when screen 120 of health management device 104. Such automatic image capture may be aided by providing indicia on health management device 104 screen 120 or elsewhere. Such indicia may be anything that allows the software of mobile device 300 to recognize that screen 120 with data displayed thereon is within capture range 304 of camera 302. Additionally, the software of mobile device 300 may recognize the shape of health management device 104, may locate display 120, and may recognize the information on display 120. Such recognition may be via recognition of visual characteristics or via logic. In addition to the indicia of device shape, logic may be programmed to recognize a symbol or bar code on the screen or housing of health management device 104. The symbol or bar code can allow the mobile device to be informed of the locations in which to find the relevant data. Alternatively, the logic may be able to interpret a series of flashes from display 120 that are unique for the device 300 or data type being presented. The software of mobile device 300 may also be able to discern relative orientation and instruct the user on adjustment (movement) necessary to align mobile device 300 with screen 120 of health management device 104. Additionally, indicia such as symbols may be used to indicate diagnostic information about the data presented on screen 120. Such diagnostic information includes, but is not limited to, indicating that at particular piece of data is suspected of being unreliable, is too high, or is too low.

Once health management device 104 and mobile device 300 are properly positioned and the image capture is completed, value 310 displayed on health management device 104 is captured by mobile device 300. Value 310 is shown as a blood glucose value. However, it should be appreciated that the blood glucose value is shown to represent all displayed data types previously discussed and information about the genesis of that data.

At this point, value 310 is within mobile device 300 as an image rather than as alphanumeric data. The image data can be sent to computer 102 or another computing device for further processing, transfer, or storage. Processing may also be performed on mobile device 300. Software within mobile device 300, or on computer 102, transforms the captured image of display 120 into alphanumeric information. By recognizing previously identified visual characteristics, or by using optical processing logic, multiple pieces of alphanumeric information can be recognized by the software on mobile device 300 (or computer 102) and extracted from the obtained image. The alphanumeric data is then saved on computer 102 or mobile device 300, although saving on mobile device 300 may be very short lived if the data is transmitted away from mobile device 300 and not needed thereon.

Once extracted, data saved on mobile device 300 can be transmitted to computer 102, 202 for further processing, transfer, or storage. If mobile device 300 is a camera phone, data is uploaded to computer 102 via the wireless communication band utilized by the phone or by making a wired connection between the phone and computer 102. Embodiments are envisioned where values are automatically wirelessly uploaded to computer 102 when obtained and processed. The software within mobile device 300 is provided with instructions on where to send the data. The data can be sent to a location where software 102 can access the data, either immediately or after a delay.

If mobile device 300 is a webcam coupled to computer 102, computer 102 has the image recognition software thereon and processes the image. If mobile device is a digital camera, computer 102 likewise has the image recognition software thereon and processes the image once the image is uploaded to computer 102. Alternatively, computer 102 can obtain an image from a webcam or camera, and then transfer the image to computer 202 for processing.

Accordingly, health management device 104 is able to electronically transmit trusted data to computer 102 when IR transmission, or another desired transmission method, is not compatible with the hardware of the user. The extracted alphanumeric data is subsequently saved on computer 102. Furthermore, the alphanumeric data may be transmitted to computer 202 and saved there as well.

While data obtained from processed images is discussed as being sent from mobile device 300 to computer 102 and from computer 102 to computer 202, it should be appreciated that data can also flow in the opposite direction. Additionally, while the specification has discussed transmitting the alphanumeric data or the image data, it should also be appreciated that both can be sent together for later verification or other purposes.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

1. A medical data transmission system including:
a processor based image capturing device including instructions thereon that when interpreted by the processor cause the processor to:
capture an image of a display of a health management device having alphanumeric medical data thereon;
perform optical character recognition on the captured image to extract the alphanumeric medical data from the image; and
save the detected alphanumeric medical data.

2. The system of claim 1, wherein the processor based image capturing device is a mobile phone.

3. The system of claim 1, wherein the instructions cause the processor to take a picture of a health management device.

4. The system of claim 3, wherein the health management device is a glucose meter.

5. The system of claim 1, wherein the alphanumeric medical data is stored on the processor based image capturing device.

6. The system of claim 1, wherein the instructions further cause the processor to wirelessly output the alphanumeric medical data to a health management program.

7. The system of claim 1, wherein the instructions cause the processor to automatically recognize that a display of a health management device is within a capture range of the image capturing device.

8. The system of claim 7, wherein the instructions further cause the processor to automatically capture the image of the display upon recognizing that the display is within the capture range.

9. The system of claim 7, wherein the instructions further cause the processor to provide direction to the user on adjustment of the relative position of the image capturing device and the health management device.

10. A computer readable medium, including operating instructions thereon such that when interpreted by a processor cause the processor to perform the steps of:
capturing an image of a display of a health management device having alphanumeric medical information thereon;
performing optical character recognition on the captured image to extract the alphanumeric medical information from the image as alphanumeric medical data; and
saving the detected alphanumeric medical data.

11. The computer readable medium of claim 10, wherein step of capturing an image automatically occurs upon detection of the display of the health management device within a capture range of a camera.

12. The computer readable medium of claim 10, wherein the saving step saves the alphanumeric medical information on a mobile phone.

13. The computer readable medium of claim 10, wherein the health management device is a glucose meter.

14. The computer readable medium of claim 10, wherein the instructions further cause the processor to perform the step of: wirelessly transmitting the alphanumeric medical data.

15. The computer readable medium of claim 10, wherein the step of wirelessly transmitting the alphanumeric medical data is performed automatically once the alphanumeric medical data is extracted.

16. A medical data transmission system including:
a wireless transceiver including a camera configured to capture an image of a display of a medical device having medical information thereon; the transceiver further including a processor having instructions thereon to analyze the image and extract the medical information from the image as alphanumeric medical data in the form of alphanumeric characters; and the transceiver further including a transmitter to wirelessly emit the alphanumeric medical data.

17. The system of claim 16, wherein the wireless transceiver is a mobile phone.

18. The system of claim 16, wherein the wireless transceiver includes instructions thereon that cause alphanumeric medical data to be wirelessly transmitted to be received by a processor based unit having health management software thereon.

19. The system of claim 18, wherein the transceiver includes instructions thereon that cause the transceiver to automatically emit the alphanumeric medical data once the alphanumeric data is extracted from the image.

20. The system of claim 16, wherein the transceiver includes instructions thereon that cause the transceiver to automatically capture the image of the display upon detection of the display within a capture range of the camera.

21. A medical data transmission system including:
a first processor based image capturing device including instructions thereon that when interpreted by the processor cause the processor to:
capture an image of a display of a health management device having alphanumeric medical data thereon; and
transmit the captured image to a second processor based device programmed to perform optical character recognition on the captured image to extract the alphanumeric medical data from the image and save the detected alphanumeric medical data.

22. The system of claim 21, wherein the first processor based image capturing device is a mobile phone.

23. The system of claim 21, wherein the instructions cause the processor to automatically take a picture of a health management device upon detection of proper orientation of the first processor based image capturing device and the health management device.

24. The system of claim 21, wherein the alphanumeric data is transmitted back to the first processor based image capturing device.

25. A medical data transmission system including:
a health management device having a outer housing including display for displaying medical data thereon, the housing further providing a first indicia thereon;
a computer readable medium having instructions thereon that, when interpreted by a processor of a processor based image capturing device cause the processor to perform the steps of:
identifying the first indicia,
capturing an image of the display of the health management device having medical data thereon; wherein the capturing is performed using information gained through the identification of the first indicia.

26. The system of claim 25, wherein the first indicia is provided on the display.

27. The system of claim 26, wherein the first indicia is a series of flashes by the display.

28. The system of claim 25, wherein the step of identifying the first indicia allows the processor to identify the device type of the health management device.

29. The system of claim 25, wherein the step of identifying the first indicia allows the processor based image capturing device to identify the orientation of the image capturing device relative to the health management device.

30. The system of claim 25, wherein the step of identifying the first indicia allows the processor based image capturing device to identify the type of medical data on the display.

31. The system of claim 25, wherein the step of identifying the first indicia allows the processor based image capturing device to identify diagnostic information about medical data on the display.
